(19) United States
(12) Patent Application Publication
(10) Pub. No.: US 2025/0253053 A1
(43) Pub. Date: Aug. 7, 2025

(54) METHOD OF USING TEMPORAL ELECTROCHEMICAL IMPEDANCE SPECTROSCOPY AND MACHINE LEARNING FOR BIOSENSING

(71) Applicants: Xiaowu (Shirley) TANG, Waterloo (CA); Tao CHEN, Waterloo (CA)

(72) Inventors: Xiaowu (Shirley) TANG, Waterloo (CA); Tao CHEN, Waterloo (CA)

(21) Appl. No.: 19/185,618

(22) Filed: Apr. 22, 2025

Related U.S. Application Data
(63) Continuation of application No. PCT/CA2024/050757, filed on Jun. 5, 2024.
(60) Provisional application No. 63/577,935, filed on Jun. 6, 2023.

(51) Int. Cl. G16H 50/30; G01N 27/02; G01N 27/327; G01N 33/68; G01N 33/74; G06N 3/0464; G06N 3/09; G06N 20/10

(52) U.S. Cl. CPC G16H 50/30; G01N 27/026; G01N 27/3278; G01N 33/6893; G01N 33/74; G06N 3/0464; G06N 3/09; G06N 20/10; G01N 2333/58

(57) ABSTRACT

A system for determining a concentration of an analyte, such as B-type natriuretic peptide (BNP), in a fluid sample, such as a blood sample, includes a sample cartridge, a sensor reader, and a data processing system. The cartridge comprises a sample chamber with an electrochemical sensor, such as a carbon nanotube thin film sensor, and a connector for coupling with the sensor reader. The sensor reader is operable to perform impedance spectroscopy sweeps on the sensor at preconfigured time intervals to generate a set of temporal impedance spectra, which is communicated to the data processing apparatus. The latter operates a trained machine learning model on the set of temporal impedance spectra to generate the concentration of the analyte in the fluid sample. The system enables convenient and reliable point-of-care analyte concentration determination for prognostication of a patient condition.

### CROSS-REFERENCE TO RELATED APPLICATIONS

This application is a bypass continuation under Section 111 (a) of PCT Patent Application No. PCT/CA2024/050757, filed on Jun. 5, 2024, which claims priority to U.S. Provisional Patent Application No. 63/577,935 filed Jun. 6, 2023, which are both incorporated by reference in their entireties including without limitation, the specification, claims, and abstract, as well as any tables and examples therein.

### BACKGROUND

Heart failure (HF) is an escalating chronic disease that affects 64.3 million people worldwide (Groenewegen, A., Rutten, F. H., Mosterd, A., Hoes, A. W., 2020. Eur. J. Heart Fail. 22, 1342-1356). HF has a high rate of hospital readmission and the associated high mortality in repeat hospitalizations, making optimal clinical management very crucial (Lin, A. H., Chin, J. C., Sicignano, N. M., Evans, A. M., 2017. Mil. Med. 182, e1932-e1937). Yet, the mechanism of HF is compensatory in nature and hence lacks specific signs in early stages (Inamdar, A. A., Inamdar, A. C., 2016. J. Clin. Med. 5, 62). Heart failure symptoms include shortness of breath, fatigue, and edema, which can be caused by many other heart and lung conditions. Biomarkers can provide a more reliable tool for ascertaining the presence of HF, its severity and treatment response. B-type natriuretic peptide (BNP), a peptide hormone secreted by the ventricular myocardium in response to myocardial wall stress, is one of the gold standard biomarkers for determining the likelihood of HF (Ezekowitz, J. A. et al., 2017. Can. J. Cardiol. 33, 1342-1433; Kim, H. N., Januzzi, J. L., 2011. Circulation 123, 2015-2019). For HF diagnosis, there are established ranges of BNP concentration to rule out and rule in HF in conjunction with other clinical information. Due to the association between an elevated BNP level and the risk of hospitalization and mortality, close monitoring of the BNP level can be used as an indicator of HF prognosis and as a guide for HF management (Ezekowitz, J. A. et al., 2017. Can. J. Cardiol. 33, 1342-1433, 2017; Wright, G. A., Struthers, A. D., 2006. Heart 92, 149-151).

The current BNP testing regimen is laborious, requiring the use of bulky and complex immunoassay equipment and highly trained professionals. The BNP testing regime can sometimes take a long time to complete, on a time scale of hours, and often requires a patient to see a phlebotomist to obtain the blood samples. However, reducing the scale and time of BNP tests for near-patient or point of care (POC) use can be difficult because the sensor or detector needs sufficient sensitivity to detect biomarkers in a clinically relevant range. For example, protein biomarkers such as BNP often have a clinically relevant range that reaches as low as pg/mL to ng/ml. Some sensors and biomarkers can be fabricated using thin film sensors, which can provide the sensitivity needed to detect protein biomarkers such as BNP. For example, nanomaterials may be used in electrochemical biosensors, and can offer high sensitivity and fast response at low production cost. One example includes carbon nanotube (CNT)-based electrochemical biosensors, which may be used as a scaffold for receptor immobilization and/or transducing elements.

While thin film sensors may be able to achieve the sensitivity to detect protein biomarkers like BNP in clinically relevant ranges (e.g., pg/L to ng/L), thin film sensors experience several drawbacks. Thin-film sensors can have sensor-to-sensor performance variability resulting from fabrication techniques and/or inherent variability in the material structure. At the nanoscale, even small variability in material structure can sometimes result in significant performance variation. For example, individual CNT and CNT network properties may vary. Differences in CNT length, tube diameter, chirality, and defects may affect the electrical and material properties of the thin films. Variation in the thin film properties can result in noisy data that inhibits the detection of an analyte. Such noise in the data can also result from the sensitivity of the thin film sensor itself, which is susceptible to perturbances in its environment.

In addition, the fabrication of thin films often requires strict process control such as the precursor concentration, catalyst deposition, growth pressure, temperature, etc. Navigating such variation to achieve reproducible and highly accurate analytical results through conventional analytical methods of protein markers requires advanced electronics and computing systems and/or high trained professionals, which limits the feasibility of applying thin film sensors to POC testing.

Accordingly, a test that can detect an analyte in clinically relevant ranges in the presence of noise is desirable.

### BRIEF DESCRIPTION OF THE DRAWINGS

Embodiments will now be described, by way of example only, with reference to the attached Figures.

FIG. 1 is a block diagram of a system for determining a concentration of an analyte in a fluid sample.

FIG. 2 is a block diagram of another system for determining a concentration of an analyte in a fluid sample.

FIG. 3 is a block diagram of a sample reader according to some embodiments.

FIG. 4 is a block diagram of another system for determining a concentration of an analyte in a fluid sample.

FIG. 5 shows a sample cartridge according to some embodiments.

FIG. 6 is a block diagram of the machine learning model architecture in some embodiments.

FIG. 7 is a block diagram for a method of determining a concentration of an analyte in a fluid sample according to some embodiments.

FIG. 8 is a block diagram of a method of training a model to determine a concentration of an analyte in a fluid sample according to some embodiments.

FIG. 9 shows four machine learning model architectures that were evaluated using receiver operating characteristics (ROC) curve using the same test set for each mode: (a) Comparison of prediction accuracy of the four models. Std: standard deviation. MSE: mean squared error. (b) Receiver operating characteristics (ROC) curve of various ML models. CNN+TD: CNN model with time dependance (TD) elements. CNN-DNN: A CNN model that does not contain fully connected layers.

FIG. 10 shows the in-sample and out-of-sample accuracy of the trained machine learning model (CNN+TD) based on confusion matrices and test validity of 4-class chamber-level classification model: (a-b) In-sample. (c-d) out-of-sample. The 4×4 matrix lists the number of sensors, with correct predictions represented by the diagonal cells and incorrect predictions represented by the off-diagonal cells. The percentage in each cell represents the proportion of corresponding classification of each true class.

FIG. 11 shows the mean and Std out-of-sample accuracy: (a) The trend of out-of-sample performance with increasing patients included in the training sample. (b) The trend of out-of-sample performance for one specific patient with increasing samples of that patient included in the training sample. Grey area: the upper and lower bounds of 95% confidence interval of the mean of out-of-sample accuracies.

FIG. 12 shows out-of-sample performance for two-class models: (a) HF Rule-out model. (b) HF Rule-in model.

FIG. 13 shows ROC curves for a rule-in model and a rule-out model: (a) In-sample and out-of-sample performance of 2-class rule-out model. (b) In-sample and out-of-sample performance of 2-class rule-in model.

### DETAILED DESCRIPTION

The present disclosure is for a system and method for determining a concentration of an analyte in a fluid sample, which in some embodiments is a blood sample. In some embodiments, the analyte is B-type natriuretic peptide (BNP), although other analytes are possible and contemplated. While the present disclosure uses determining the likelihood of heart failure as an example application for detecting and classifying BNP concentrations in blood samples, the systems and methods described in the disclosure may be directed to any application in which a measurement of the concentration of BNP or other analyte in a fluid sample is desired.

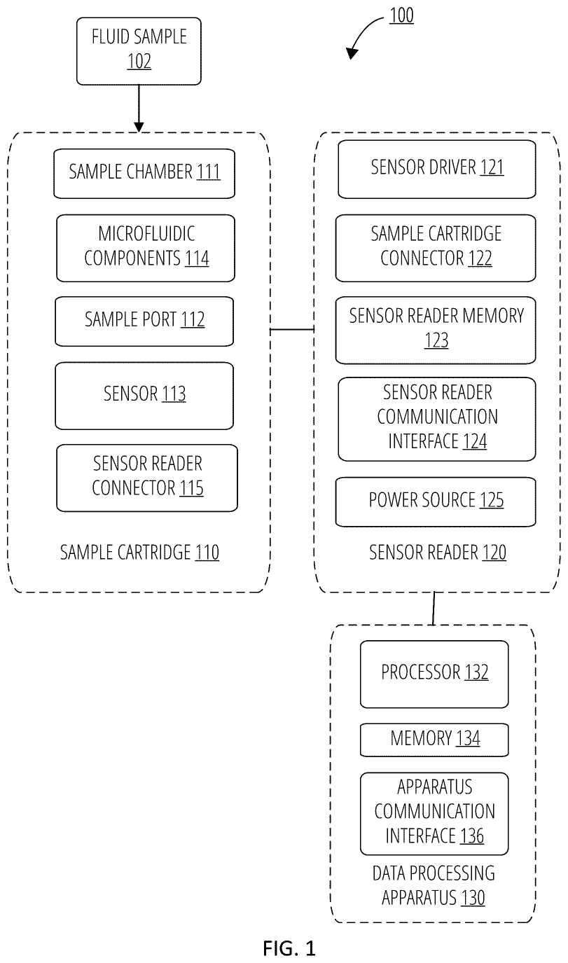

FIG. 1 is a block diagram of a system for determining a concentration of an analyte, such as protein biomarker like B-type natriuretic peptide (BNP), in a fluid sample 102. The system 100 includes a sample cartridge 110, sensor reader 120, and a data processing apparatus 130, such as a computer or other computing device, in communication with the sensor reader 120 using a sensor reader communication interface 124 and an apparatus communication interface 136. As will be discussed in further detail below, the sample cartridge 110 receives a fluid sample 102 and transports the fluid sample 102 to a sample chamber 111 for contact of the fluid sample 102 with at least one sensor 113. The sensor reader 120 performs an impedance spectroscopy sweep on the sensor(s) 113 to generate temporal impedance spectra. In some embodiments, the sensor reader 120 performs the impedance spectroscopy sweep in combination with the data processing apparatus 130, while in other embodiments the sensor reader 120 performs the impedance spectroscopy sweep alone. The data processing apparatus 130 has a processor 132 and a memory 134 storing instructions operable by the processor 132 to process the temporal impedance spectra using at least one trained machine learning model to determine the concentration of the analyte and/or classify the concentration of the analyte into a class for, e.g., diagnostic purposes. In some embodiments, the analyte may be BNP, the fluid sample may be a blood sample, and the concentration of BNP may be determined and/or classified for determining the likelihood of heart failure in the patient who provided the blood sample.

The sample cartridge 110 may have a sample port 112 to receive a fluid sample 102. The sample port 112 may be connected to microfluidic components 114 that transport the fluid sample 102 from the sample port 112 to at least one sample chamber 111. The microfluidic components 114 transport the fluid sample to the sample chamber(s) 111 by any suitable means for delivering the fluid sample 102 to sample chamber(s) 111. In some embodiments, the microfluidic components 114 may include microchannels. The sample chamber 111 has or is coupled with one or more sensors 113 and may be positioned or coupled to the microfluidic components 114 such that the fluid sample 102 is directed to at least one surface of the sensor(s) 113.

In some embodiments, the sensor(s) 113 are thin film sensors. The thin film sensors may be composed of carbon nanotube thin films (CNT-TFs) or any other suitable thin film sensor for detecting an analyte. The carbon nanotube thin films may be functionalized to bind the analyte. For example, the CNT-TFs may be functionalized to bind BNP by immobilizing monoclonal anti-BNP antibodies on the CNT-TF surface.

The sample cartridge 110 may include a sensor reader connector 115 that is electrically coupled to at least one of the sensors 113. The electrical coupling may include electrodes coupled to the sensor(s) 113 in a manner suitable for performing impedance spectroscopy measurements, and further coupled to or integrated in the sensor reader connector 115 on the sample cartridge 110. For example, a thin film sensor, or each thin film sensor, may be connected to at least two electrodes for performing electrochemical impedance spectroscopy (EIS). The electrodes may be fabricated or connected to the sensor by any suitable means for the sensor type and/or in a configuration suitable for allowing the sensor reader connector 115 to electrically couple with the sample cartridge connector 122 on the sensor reader 120. The sample cartridge 110 may be configured to receive a single fluid sample or multiple fluid samples for concurrent and/or sequential analysis.

The sensor reader 120 may include a sensor driver 121, a sensor reader memory 123, a sensor reader communication interface 124, and a power source 125. The sensor driver 121 may be powered by the power source 125, and may be operable to perform the impedance spectroscopy sweep as described, and to store the impedance spectrum data in the sensor reader memory 123. The sensor reader 120 may have a sensor reader communication interface 124 coupled to the sensor reader memory 123 to communicate the impedance spectrum data to a data processing apparatus 130 and/or to receive configurations commands from the data processing apparatus 130 to control the parameters of the impedance spectroscopy sweeps. The sensor reader communication interface 124 may be any suitable communication interface for communicating with the data processing apparatus 130. In different embodiments, the sensor reader communication interface 124 is wired (e.g., USB) or wireless (e.g., WiFi, 5G) interface, and may include or cooperate with circuitry for performing wired or wireless communication with the data processing apparatus 130, which may be directly with the apparatus communication interface 136, or may be through a network, which may be wired or wireless, which may be the Internet, as appropriate. In different embodiments, the apparatus communication interface 136 likewise takes any of the foregoing forms, so long as the sensor reader communication interface 124 and the apparatus communication interface 136 are mutually configured for the intercommunications described herein.

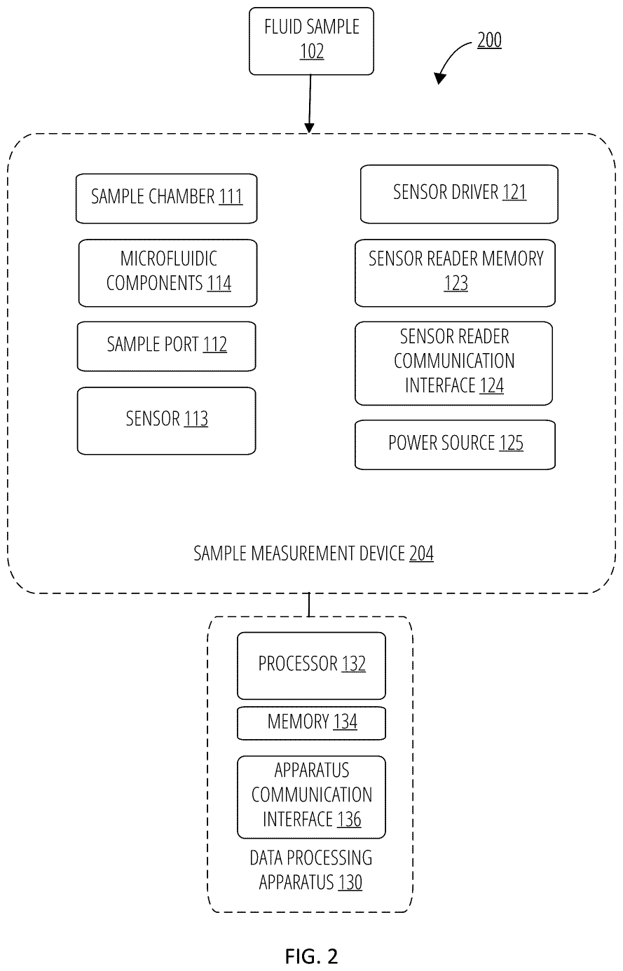

In some embodiments, the sensor reader 120 and sample cartridge 110 are separate components such that the sensor reader 120 is operable to collect temporal impedance spectra for more than one fluid sample 102. Alternatively, FIG. 2 shows a block diagram of another system 200 for determining a concentration of an analyte in a fluid sample according to an embodiment of the present disclosure. System 200 is substantially similar to system 100, except in that the above-described components of the sample cartridge 110 and the sensor reader 120 are present in a single sample measurement device 204. In such cases, the device 204 may not require a sensor reader connector 115 or a sample cartridge connector 122, and instead the sensor driver 121 may be connected directly with the at least one sensor 113 within the device 204. Other embodiments are possible and contemplated wherein the features and functionalities described herein are possessed and provided by any number of cooperating devices and structures.

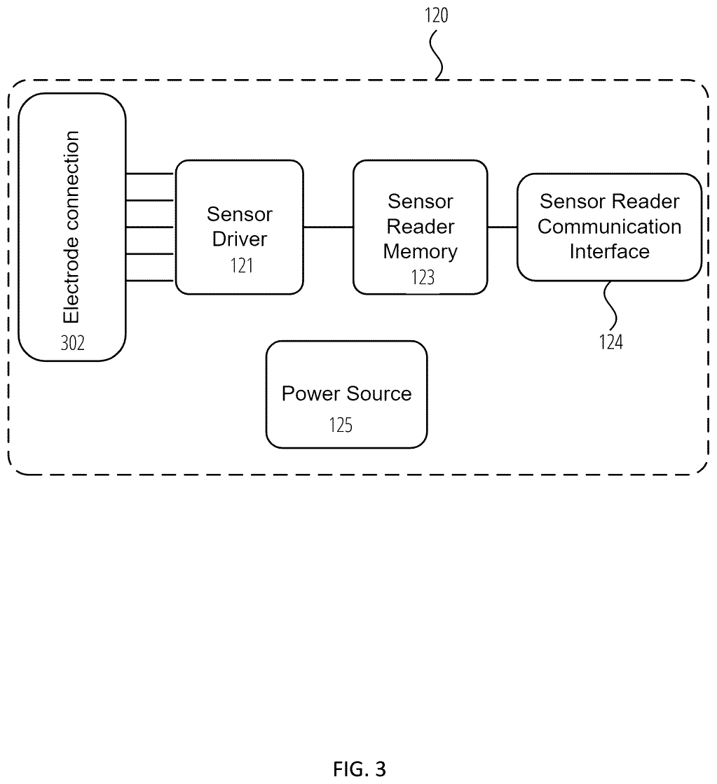

FIG. 3 shows an embodiment of the sensor reader 120. The sample reader 120 may have a sample cartridge connector 122 that connects to the sample reader connector 115 on the sample cartridge 110 to electrically couple the sample cartridge 110 and the sensor reader 120. In this example, the sample cartridge connector 122 may include or be embodied as electrode connections 302. The sample reader also has a sensor driver 121 operable to perform an impedance spectroscopy sweep on one or more sensors. The sensor driver 121 may be or include a potentiostat. The sensor driver 121 may be operable to perform impedance spectroscopy measurements at multiple time intervals to generate a set of temporal impedance spectra. The impedance spectra and/or temporal impedance spectra may be stored on a sensor reader memory 123 that is connected to the sensor driver 121. The sensor reader memory 123 and the sensor driver 121 may be powered by a power source 125. The power source 125 may be a battery included in the sensor reader 120, for example, to enable use of the sensor reader 120 as a handheld device for POC use. The sensor reader 120 may be powered by an external power source 125 in other embodiments. The sensor reader 120 may have a sensor reader communication interface 124 coupled to the sensor reader memory 123 to communicate the impedance spectrum data to a data processing apparatus 130 and/or to receive configurations commands from the data processing apparatus 130 to control the parameters of the impedance spectroscopy sweeps. The sensor reader 120 may have a housing 126 enclosing some or all of the sensor reader 120 components, such as the sensor driver 121, the power source 125, and/or the sensor reader memory 123.

As indicated above, in some embodiments the sensor reader 120 is operable to perform the impedance spectroscopy sweeps on the at least one sensor 113 in combination with the data processing apparatus 130, while in communicative contact with the data processing apparatus 130, while in other embodiments the sensor reader 120 is operable to perform the impedance spectroscopy sweeps on the at least one sensor 113 alone, that is not in communicative contact with the data processing apparatus 130.

In the former case, the sensor driver 121 may be or include a potentiostat, and the processor 132 of the data processing apparatus 130 may be configured, or the memory 134 of the data processing apparatus 130 may store instructions operable by the processor 132, to control the sensor driver 121 via the apparatus communication interface 136 and the sensor reader communication interface 124 to perform the impedance spectroscopy sweep on the at least one sensor 113. In some embodiments, the collected temporal impedance spectra are stored in the sensor reader memory 123 and then communicated to the data processing apparatus 130, while in other embodiments the collected temporal impedance spectra are communicated to the data processing apparatus 130 directly during the performance of the measurements.

In the latter case—that is, the sensor reader 120 is operable to perform the impedance spectroscopy sweeps on the at least one sensor 113 alone—the sensor driver 121 may include a potentiostat and further include a processor or other circuitry operable to perform the impedance spectroscopy sweeps on the at least one sensor 113 as described while the sensor reader 120 is out of communicative contact with the data processing apparatus 130. In some embodiments, the sensor reader memory 123 store instructions performable by the sensor driver 121 processor to perform the impedance spectroscopy sweep. In any event, the sensor driver 121 may be operable to store the collected temporal impedance spectra in the sensor reader memory 123 for later communication to the data processing apparatus 130 via the apparatus communication interface 136 and the sensor reader communication interface 124. In some embodiments, the sensor reader 120, while communicatively connected with the data processing apparatus 130, may be configurable with respect to one or more aspects of the functionality of the sensor reader 120 as described herein, include without limitation any parameters of the impedance spectroscopy sweeps. Such configuration may involve modification of conditions of the sensor driver 121—when, for example, the sensor driver 121 includes a processor with or connected with programmable read-only-memory containing instructions performable by the processor—or the instructions stored in the sensor reader memory 123 for performance by the sensor driver 121.

In some embodiments of the foregoing embodiments, the sensor reader 120 may be operable independently of the data processing apparatus 130 during collection of the temporal impedance spectra as described. For example, in some embodiments the sensor reader 120 has a power source 125 operable to power the sensor driver 121 and sensor reader memory 123 to perform the impedance spectroscopy sweeps and to store the collected temporal impedance spectra in the sensor reader memory 123. The sensor reader 120 may have an actuator, such as a push button of a switch, selectively operable by a user for activating the sensor reader 120 to perform the impedance spectroscopy sweeps. Thus, such embodiments may be especially useful and convenient for point-of-care use, inasmuch as a user may be enabled to collected a fluid sample 102 in the form of a blood sample or other body fluid sample at bedside, charge a sample cartridge 110 as described, couple the charged sample cartridge 110 with the sensor reader 120 as described, and perform the impedance spectroscopy sweeps on the fluid sample 102 without or with minimal delay from the original fluid sample 102 collection and/or contact with the at least one sensor 113. Then, the sample reader 120 may be communicatively coupled with the data processing apparatus 130 as described for processing of the collected temporal impedance spectra as described. This may be done immediately, or with minimal delay, by any of the communication connection means as described herein. Or, alternatively, it may be done some period later, when convenient, inasmuch as the temporal impedance spectra are already collected and safely stored in the sensor reader memory 123.

Some embodiments of the foregoing embodiments may be operable as a handheld device. For example, the sensor reader 120 may have a largest dimension of 10 cm or less (e.g., 7.5 cm×4.8 cm, by 1.9 cm), and the sample cartridge 110 may have a largest dimension of 3 cm or less (e.g., 2.2 cm×2.9 cm×0.9 cm).

Thus, the various embodiments of the techniques disclosed herein provide many options for collecting and performing impedance spectroscopy measurements of collected fluid samples and point-of-care for processing and prognostication, suitable for a diversity of environments and situations.

The impedance spectroscopy sweeps may be performed at a preconfigured number of time intervals, and within a preconfigured period of time. In different embodiments, the impedance spectroscopy sweeps are performed in any appropriate number of time intervals, and may be performed within any appropriate total time period, depending on the nature of the analyte, the fluid, and the sensor(s). In some embodiments, the number of time intervals and/or total time period may be selected according to the kinetic process of an analyte-receptor interaction and/or any other relevant physiological event or phenomenon. For example, where the analyte is BNP, the fluid sample is a blood sample, and the sensor(s) are functionalized as described above, the impedance spectroscopy sweeps may be performed repeatedly from about 0 minutes (i.e., exposure of the sensor(s) by the blood sample) to about 45 minutes using 8 time intervals, or to about 15 minutes using 4 time intervals. In some embodiments, the total time period may be about 300 minutes. Each impedance spectroscopy sweep may be performed at any appropriate number of different frequencies, which in different embodiments may be between about 0.1 Hz to about 100 KHz. In some embodiments, the number of different frequencies may be 10. In some embodiments, the frequency range may be 780 Hz to 10 kHz. In other embodiments, the frequency range may be 80 Hz to 10 KHz. Alternatives to the foregoing are possible and contemplated.

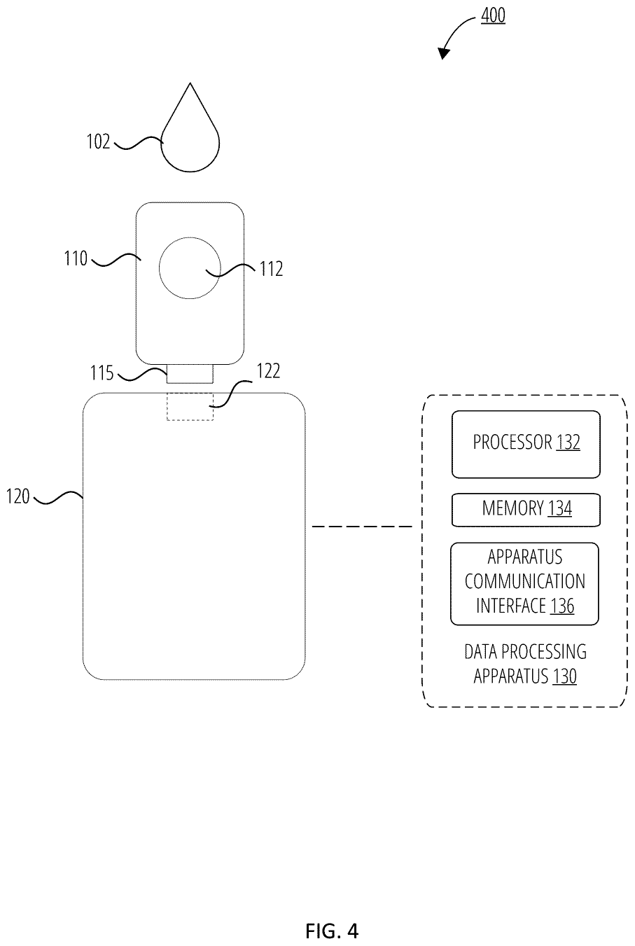

FIG. 4 is an example system 400 for detecting an analyte according to an embodiment. System 400 may be substantially similar to system 100. System 400 may receive a fluid sample 102 at a sample port 112 of a sample cartridge 110. The sensor reader 120 may include a sensor driver 121, a sensor reader memory 123, a sensor reader communication interface 124, and a power source 125. The sensor reader 120 may have a sensor reader communication interface 124 coupled to the sensor reader memory 123 to communicate the impedance spectrum data to a data processing apparatus 130 and/or to receive configurations commands from the data processing apparatus 130 to control the parameters of the impedance spectroscopy sweeps. The sample cartridge 110 may be configured to connect to the sensor reader 120 via a sensor reader connector 115 coupled to a sample cartridge connector 122 to electrically couple the sensor driver 121 to the sensor(s) 113.

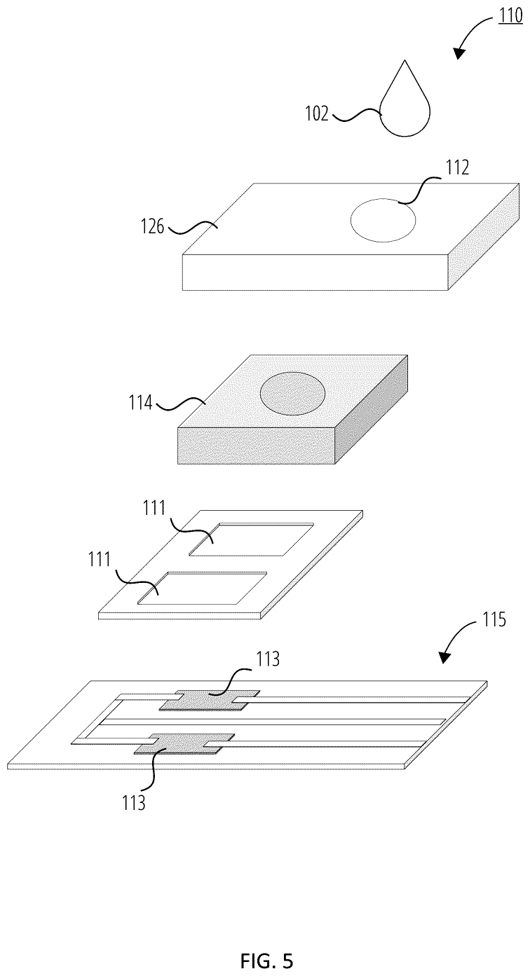

FIG. 5 shows the sample cartridge 110 according to another embodiment. The sample cartridge 110 may have a sample port 112 to receive a fluid sample 102. The sample port 112 may be connected to microfluidic components 114 that transport the fluid sample 102 from the sample port 112 to at least one sample chamber 111. The microfluidic components 114 transport the fluid sample to the sample chambers 111 by any suitable means for delivering the fluid sample 102 to sample chambers 111. In some embodiments, the microfluidic components 114 may include microchannels. The sample chamber 111 has or is coupled with one or more sensors 113 and may be positioned or coupled to the microfluidic components 114 such that the fluid sample 102 is directed to at least one surface of the sensors 113.

The sample cartridge 110 may include a sensor reader connector 115 that is electrically coupled to the sensors 113. The electrical coupling may include having electrodes coupled to the sensor(s) 113 in a manner for performing impedance spectroscopy using a sensor reader 120 (not shown in FIG. 5), and further coupled to or integrated in the sensor reader connector 115 on the sample cartridge 110. For example, a thin film sensor, or each thin film sensor, may be connected to two electrodes for performing electrochemical impedance spectroscopy (EIS). In the embodiment shown in FIG. 5 the electrodes may be patterned onto the substrate and over the sensors 113 and extend along the substrate to form the sensor reader connector 115, which electrically couples with the sample cartridge connector 122 on the sensor reader 120.

As described above, the data processing apparatus 130 has a processor 132 and a memory 134 storing instructions operable by the processor 132 to process the impedance spectra using at least one trained machine learning model to determine the concentration of an analyte in a fluid sample. In some embodiments, the analyte is BNP, the fluid sample is a blood sample of a patient, and the data processing apparatus 130 uses the at least one trained machine learning model to determine the concentration of BNP in the blood sample and/or to classify the concentration of the BNP into a class for assessing the likelihood of heart failure in the patient.

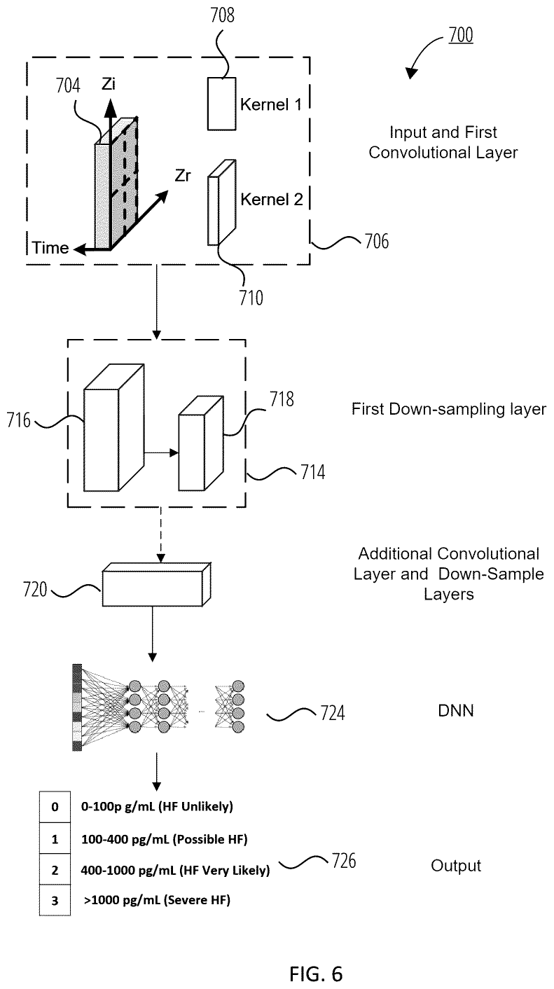

FIG. 6 is a block diagram of an embodiment of a trained machine learning model 700 architecture that may be used by the apparatus 130 to determine, generate, and/or classify the concentration of an analyte in a fluid sample. While the block diagram shows an output involving specifically a concentration of BNP in a blood sample, this example is non-limiting, and the principles described herein are applicable for any analyte and fluid sample. The machine learning model may be a supervised learning model. The machine learning model may be trained using the temporal impedance spectra as features of the input layer 704. In the embodiment shown in FIG. 6, the input layer may include K features comprising the real impedance measurements (Zr) and the imaginary impedance measurements (Zi) under a number of frequencies, with measurements collected before and after adding BNP to the sensor(s), and at a number of time points in a number of time intervals as described. Other suitable inputs may be used to train the machine learning model, such as dry-state resistance, characteristics or parameters of the device, time stamps, sensor manufacturing/fabrication parameters, and/or any other parameters pertaining to the device or sensor. The raw data of the impedance spectroscopy measurements may be configured into a matrix for training and/or processing by the machine learning model.

The machine learning model architecture may have at least one convolutional layer 706 as a hidden layer that applies at least one kernel 708 or group of kernels exhausting all linear combinations within each of the temporal impedance spectra, i.e., exhausting all linear combinations of local Zi and Zr, capturing the change of Zi and Zr at one time point. The convolutional layer may have a second kernel 710 or second group of kernels exhausting all linear combinations of features across the set of temporal impedance spectra, i.e., exhausting all linear combinations of each feature across different time points. The second kernel 710 may represent the time dependence of the model.

The machine learning model architecture may have at least one down-sampling convolutional layer 714 applied to the intermediate data 316 resulting from the first convolutional layer. The down-sampling convolution layer may be a max pooling layer, which extracts the local maximums and minimums in each dimension, which reduces the size of the intermediate data and can smooth noise present in the data.

The machine learning model architecture may include additional convolutional layers and/or down-sampling layers. For example, in FIG. 6, a second convolutional layer and a second max pooling layer are included to reduce the second set of intermediate data 318 output by the max pooling layer into a vector 720. The vector 720 may be input into a deep neural network (DNN 724) layer. The DNN 724 structure may be fully connected or partially connected. In the example shown in FIG. 6, the DNN is fully connected.

The DNN 724 architecture may be expressed as follows: let f (Inputs) denote the structure that provides the corresponding BNP concentration class given the input features, and let f^{DNN} (Inputs; W) denote a deep neural network representation of f(Inputs) given the weights W. Then f^{DNN} (Inputs; W) has the expression as follows with L hidden layers:

$${f}^{DNN} ( Inputs ; W ) = W out ∘ S ∘ WL ∘ ReLU ∘ … ∘ ReLU ∘ W 1 ∘ ReLU ∘ Win ∘ Inputs .$$

The weights may be unknown at first, and supervised network training may be used to determine the weights according to the following loss function:

$$Loss ( \tilde{W} ) = \frac{1}{n} {\sum }_{i = 1}^{n} {[ {Class}_{i} - {f}^{DNN} ( {Inputs}_{i} ; \tilde{W} ) ]}^{2} ,$$

where n represents the sample size, and Class; and Inputs; are the true BNP concentration class and the corresponding inputs, respectively, for sample i. The training can provide an estimated network for classification, denoted by \hat{f}(Inputs), with the estimated weights Ŵ as such

\hat{f}(Inputs):=f^{DNN}(Inputs; Ŵ).

To find the desired weights based on a given DNN structure, a sufficiently large number of training epochs may be implemented to update the weights for optimization. During such updates, overfitting can occur when the DNN 724 learns not only from the true signals but also from the noise. This may cause the model to have a high in-sample prediction accuracy but lower out-of-sample performance, where in-sample prediction accuracy refers to the model's performance with respect to the training data and out-of-sample prediction accuracy refers to the machine learning model's performance with respect to new data or data outside of the training data. To avoid overfitting, a holdout method can be applied where the sample is randomly split into two groups in each epoch, for example, 80% as the training data to find an estimator f (Inputs) that minimizes the training loss, and 20% as the validation data to monitor the out-of-sample predicting accuracy of the estimated network. Gaussian dropouts and/or Gaussian noises can be implemented in each epoch, which may include randomly dropping the neurons from the network and adding noise to the training. Such stochastic regularization techniques may improve the network's robustness and reduce over-fitting. This approach involves finding the epoch, and hence the corresponding \hat{f}(Inputs), where the estimated network provides the best validation (i.e., out-of-sample) prediction accuracy while ensuring that the gap between the training and validation accuracies remains sufficiently small.

Note that prior to this weight estimation, the model specification may be determined, including the selection of the number of hidden layers, the number of neurons in each hidden layer, and the parameters for the Gaussian dropouts and Gaussian noises (which may simulate variability in the data). The model specification may be configured to increase the model's performance in response to unseen data and/or to reduce overfitting of the model during training. In some embodiments, 10-fold cross-validation may be applied on candidate model structures, where the data is split up into ten subsets (folds) and the holdout method was repeated ten times with each subset being the validation set for evaluating the out-of-sample performance of the model trained by the remaining nine subsets. In the candidate models, 20% Gaussian dropouts may be applied to or right after the input layer, 50% dropouts to the rest but the last hidden layer, and zero-mean Gaussian noises with standard deviations ranging from 0.1 to 0.5 may be added to the input and hidden layers (except the last hidden layer). The structure with the highest average validation accuracy over all ten folds may be selected for the final DNN training.

The activation function for the machine learning model may include a combination of the rectified linear unit (ReLU) and the sigmoid (logistic) function for different layers of the model. The ReLU activation function is defined as the positive part of its argument (ReLU(·)=max (0,·)) and is known to perform well for classification applications. However, the classification outputs of the ReLU can be less intuitive to interpret because the ReLU has a range of zero to infinity. The sigmoid function has a range of 0 to 1 (S(x)=(1+e^{-x})⁻¹) and can be used for outputs that are probabilities, which is sometime more intuitive to interpret. In the sample machine learning model shown in FIG. 6, the sigmoid activation function may be used for the last hidden layer to generate the classification outputs, i.e., the BNP concentration and/or categorization into one of the rule-in or rule out classes for determining the likelihood of heart failure, and the ReLU activation function may be used for all preceding layers in the model, including the remaining hidden layers and the input layer of the machine learning model.

The output 726 of the trained machine learning model may be a concentration, characteristic, or other parameter of the analyte and/or a classification of the analyte parameter or concentration into a class. The class may be any class suitable for a particular analyte for an application. In some embodiments, the class may be any class suitable for classifying the likelihood of a disease with respect to a BNP concentration. In the example machine learning model architecture shown in FIG. 6, the trained machine learning model may output a classification of the BNP concentration ([BNP]) according to one of the following four classes: Class 0 if [BNP]=0-100 μg/mL, unlikely HF; Class 1 if [BNP]=100-400 pg/L, possible HF; Class 2 if [BNP]=400-1000 μg/mL, HF very likely; and Class 3 if [BNP]>1000 μg/mL, severe HF. The trained machine learning model may classify the BNP concentration according to a two-class rule-out and/or rule-in model. In the rule-out model, Class 0 may be considered 0 or negative and Classes 1-3 may be considered 1 or positive. In the rule-in model, Classes 0 and 1 may be considered 0 or negative, and Classes 2 and 3 may be considered 1 or positive. Alternative classifications are possible and contemplated.

A typical biosensor may detect the concentration of an analyte by measuring signals generated due to biological or chemical reactions proportional to analyte concentration. However, this method may not be effective for systems where classification of the measured signal changes between classes within one standard deviation. This problem can occur, for example, when detecting, measuring, and classifying BNP concentrations. Using a trained machine learning model 700 may overcome such problems related to the variance in sensors, devices, blood samples, etc., and provide a method and system for generating and classifying the concentration of an analyte in a fluid sample.

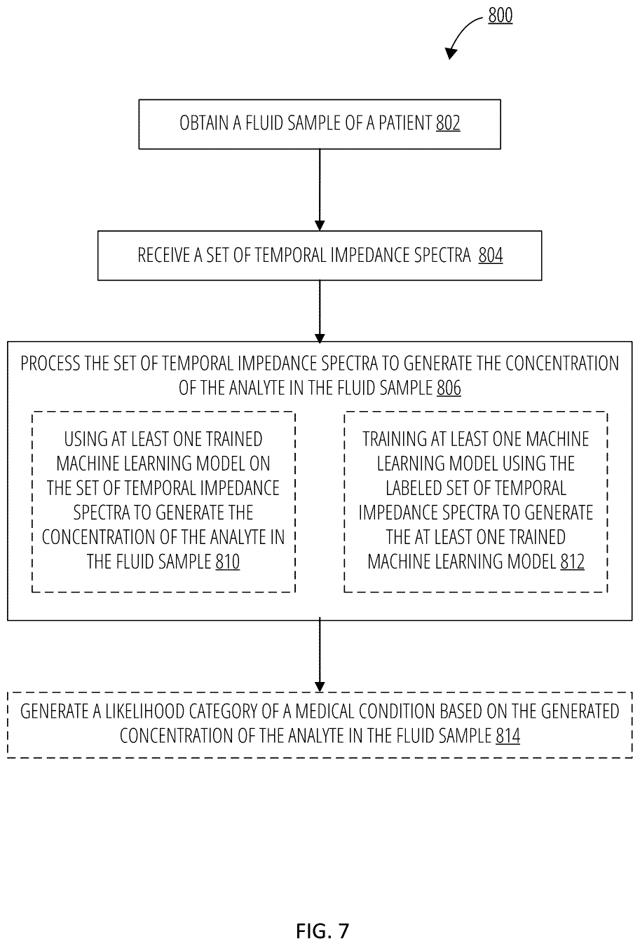

FIG. 7 is a block diagram for a method 800 of determining a concentration of an analyte in a fluid sample 102 according to an embodiment. In some embodiments, the analyte is BNP and the fluid sample is a blood sample of a patient.

At 802, a fluid sample is obtained, which may be a fluid sample of a patient. More than one fluid sample may be obtained, and where the fluid samples are fluid samples of patients, more than one fluid sample may be obtained from the same patient or from different patients. For example, obtaining more than one fluid sample at different times may be used to assess the self-learning capacity of the trained machine learning model 700. Blood samples from multiple patients may be used to generate labelled data for training a machine learning model for determining the concentration of BNP in blood. Obtaining a blood sample from a patient may be used to determine the likelihood of heart failure in the patient or any other medical condition that may be informed by the concentration of BNP in a blood sample.

At 804, a set of temporal impedance spectra may be received. The temporal impedance spectra may be generated by performing an impedance spectroscopy sweep on at least one sensor at a plurality of time intervals.

At 806, the set of temporal impedance spectra may be processed to generate the concentration of the analyte in the fluid sample. At 806, processing the set of temporal impedance spectra may further include using at least one trained machine learning model on the set of temporal impedance spectra to generate the concentration of the analyte in the fluid sample 102.

At 810, the trained machine learning model (e.g., trained machine learning model 700) may include at least one convolutional neural network. The convolutional neural network may include at least one convolutional layer applying at least one kernel 708 exhausting all linear combinations within each of the temporal impedance spectra and/or at least one kernel 710 exhausting all linear combinations of features across the set of temporal impedance spectra.

At 810, the trained machine learning model 700 may include at least one down-sampling convolutional layer after the at least one convolutional layer, such as a max pooling layer.

At 810, the trained machine learning model 700 may include at least one deep convolutional neural layer or deep neural network. The deep neural network may have fully connected layers after the at least one down-sampling convolution layer.

At 812, the trained machine learning model may be trained by obtaining at least one labelled set of temporal impedance spectra, which are characteristic of the sensor exposed to the blood sample, and training the machine learning model using the labeled set of temporal impedance spectra to generate the at least one trained machine learning model.

At 814, a likelihood category of a medical condition, such as heart failure, may be generated based on the generated concentration of the analyte, such as BNP, in the blood sample. For example, in the case of generating the likelihood of heart failure, the BNP concentration may be classified according to a 4-class model, a 2-class rule-in model, and/or a 2-class rule out model, as described elsewhere in the present disclosure. In some embodiments, the generated likelihood category of the analyte concentration, characteristic, or parameter may be any other suitable classification output for a given application.

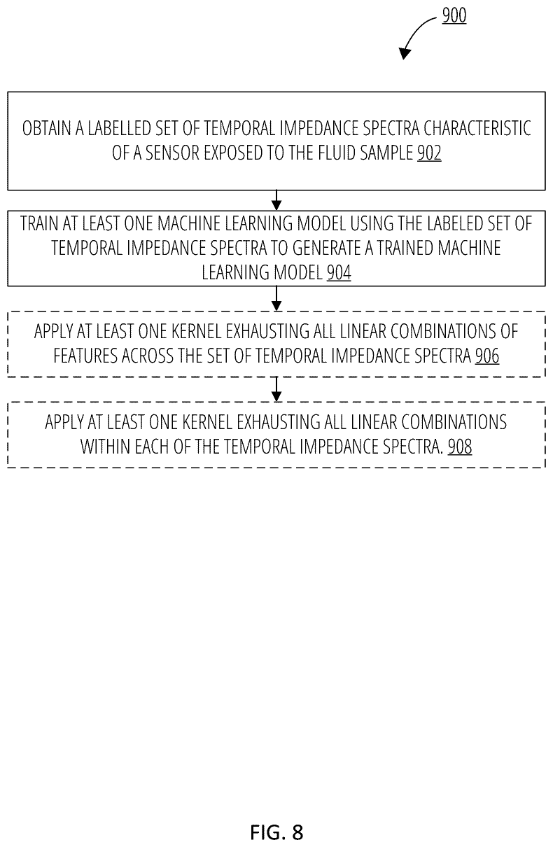

FIG. 8 is a block diagram of a method 900 of training a model to determine a concentration of an analyte in a fluid sample according to an embodiment. At 902, a labelled set of temporal impedance spectra is obtained, which may be characteristic of a sensor exposed to a fluid sample 102. At 904, a machine learning model is trained using the labeled set of temporal impedance spectra to generate a trained machine learning model (such as trained machine learning model 700). The trained machine learning model 700 may include at least one convolutional neural network (CNN)

At 906, the CNN may apply at least one kernel 708 exhausting all linear combinations within each of the temporal impedance spectra.

At 908, the CNN may apply at least one kernel (e.g., a second kernel 710) exhausting all linear combinations of features across the set of temporal impedance spectra.

The trained machine learning model 700 may include at least one down-sampling convolutional layer after the at least one convolutional layer, which may be, for example, a max pooling layer. The trained machine learning model 700 and/or the convolutional neural network may include at least one deep convolutional neural layer, which may have fully connected or partially connected layers.

To gain a better understanding of the present disclosure, the following examples are set forth. These examples are for illustrative purposes only.

### Example 1—Comparing Performance of Different Machine Learning Model Architectures

Based on the machine learning model 700 architecture shown in FIG. 6, at least four different machine learning models are possible according to different combinations of the layers described above: (1) a “CNN” model 1004 including all layers and elements except for the second kernel/group of kernels 710 containing the time dependence; (2) a “DNN” model 1008 including only the DNN structure described above without the CNN layers; a “CNN-DNN” model 1006 including all layers and elements except for the DNN structure; and (4) a “CNN+TD” 1002 model including at least all features in the trained machine learning model 700 as shown in FIG. 6 and described above. In some embodiments, the machine learning model may be self-learning and use data from new blood samples blood sample 102 to further train the model.

A comparison of these models shows that the CNN+TD model 1002 can have the highest performance with respect to determining BNP concentration. A data set containing labelled BNP concentrations associated with blood samples 102 was divided into a training data set (90%) and a test data set (10%). The same training set was used for each of the four different machine learning models noted above to compare the mean, standard deviation (Std), and mean squared error (MSE) of accuracies using laboratory results as references. The comparison of these parameters between the four different machine learning model architectures is shown in Table 1.

TABLE 1
Comparison of the prediction accuracy
of the four machine learning models

| | DNN | CNN | CNN − DNN | CNN + TD |
|---|---|---|---|---|
| Mean | 51.47% | 59.66% | 61.63% | 62.13% |
| Std | 26.48% | 23.76% | 18.98% | 19.05% |
| MSE | 0.1948 | 0.2995 | 0.3438 | 0.3497 |

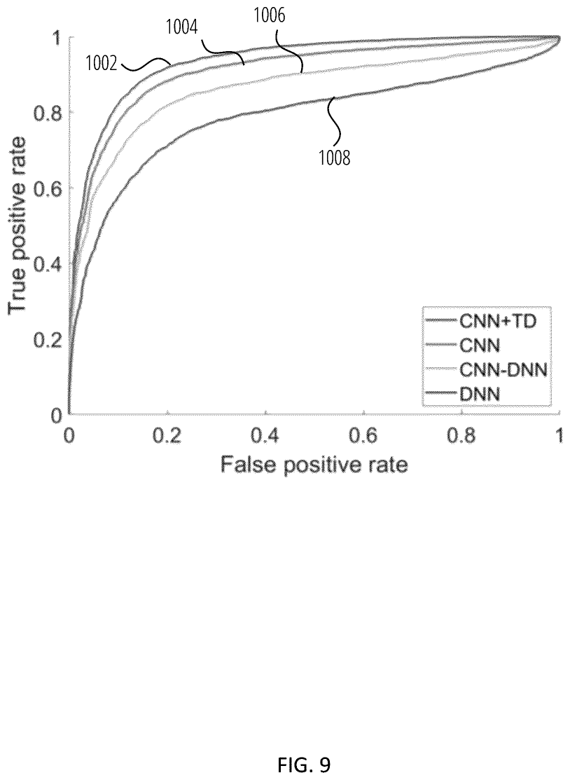

In addition, the four machine learning model architectures were evaluated using receiver operating characteristics (ROC) curve using the same test set for each mode, which is shown in FIG. 9. The true positive rate (TPR) was calculated as true positive cases (BNP level in Class 2 and 3 and predicted as Class 2 and 3 of the 4-class model) divided by true positive cases and false negative cases (BNP in Class 2 and 3 but predicted as Class 0). The false positive rate (FPR) was calculated as false positive cases (BNP level in Class 0 but predicted as Class 2 and 3 of the 4-class model) divided by false positive cases and true negative cases (BNP in Class 0 and predicted as Class 0). BNP levels in class 1 or predicted as class 1 were not included in this calculation because BNP level in this range is neither interpreted as HF highly likely or highly unlikely. The areas under curve (AUC) of the DNN, CNN-DNN, CNN and CNN+TD models were 0.6087, 0.7415, 0.7918, and 0.8427 (Table 1), respectively, indicating their accuracies in comparison with laboratory BNP results. There may be an increasing trend of AUC when moving from a fully connected structure to a locally connected structure. Among the four models, the CNN+TD 1002 model can perform better than the other models, with the highest prediction accuracy and AUC in the ROC analysis. In this example, the ROC was evaluated as compared to a laboratory BNP immunoassay which may not necessarily represent the “true” level of BNP.

### Example 2—In-Sample and Out-of-Sample Accuracy

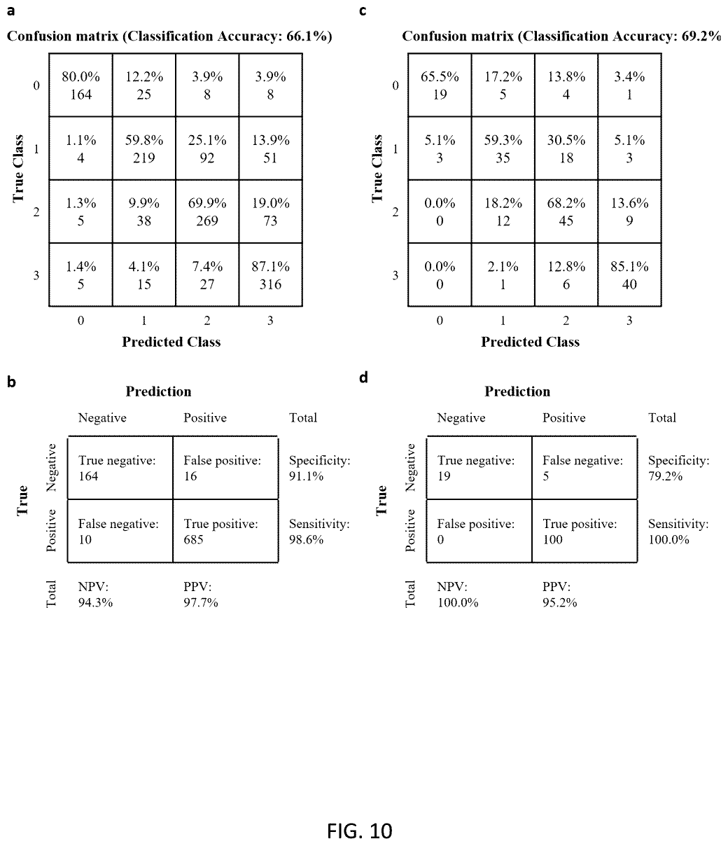

FIG. 10 shows the in-sample and out-of-sample accuracy of the trained machine learning model (CNN+TD). The in-sample test refers to prediction of BNP class using data used for model training, whereas out-of-sample test uses data that the model has never seen. FIG. 10a and FIG. 10c show the classification accuracy of the trained machine learning model for the in-sample (FIG. 10a) and out-of-sample (FIG. 10c) prediction accuracies of the trained machine learning model's interpretation of the temporal impedance spectra performed on the sensor in the sample chamber with a blood sample.

The test sensitivity, specificity, positive predictive value (PPV) and negative predicted value (NPV) of the test is shown in FIG. 10b and FIG. 10d based on a comparison between the system and a standard BNP test performed in a centralized laboratory. For the in-sample test (FIG. 10b), among all samples of BNP 0-100 pg/mL (class 0; labelled negative) as measured by a laboratory test, 91.1% were correctly classified as class 0 by the model; among all samples of BNP>400 pg/mL (class 2 and 3; labelled positive), 98.6% were correctly classified as positive. Among those classified as negative (or positive), 94.3% and 97.7% were measured to be negative (or positive). FIG. 10d shows the corresponding measurements for the out-of-sample tests.

The gap between the overall training and blind test accuracies is around 3.1 percentage points, which may indicate the model is not subject to overfitting. Thus, the accuracy for patient samples apart from training sample may be expected to approach the overall out-of-sample accuracy. This training-testing gap may be expected to further reduce under larger samples, where the model can be further optimized over a more general pool of candidates. In addition, the accuracies of class 1 and class 2 appear to be lower than that in other two classes, which may indicate class 0 and class 3 are captured by the trained machine learning model more precisely. The gap between the overall accuracies for class 1 and class 3 is around 27.3 percentage points. More samples from class 1 and class 2 may improve the prediction accuracy of the trained machine learning model 700.

### Example 3—Self Learning Capacity of the Trained Machine Learning Model

To explore the trend of the out-of-sample performance of the 4-class sensor-level classification model with increasing sample size, the following experiment was performed:

(1) Divide the data into two parts: (a) one patient that is randomly chosen; and (b) the rest of patient samples aside from the particular patient.

(2) Given a sample size N (N=30, 40, 50, 60, 70, 80, 90, 100, 110, 120, 130, 134, 150, 160, 170, 180), N samples were randomly extracted from (b) to form subsample (c). Build a model with fixed CNN+TD structure considering (c) as the training samples.

(3) Calculate the out-of-sample accuracy considering (a) as the test sample.

(4) Repeat Steps 1-3 fifty times as simulation.

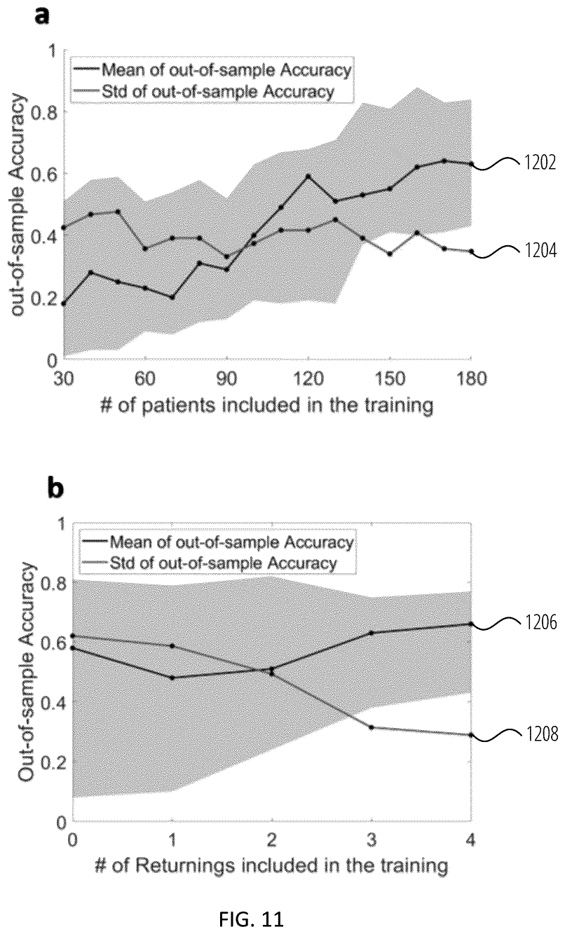

Referring to FIG. 11, this experiment can show the simulated average BNP prediction accuracies of the machine learning model under the training sample size N. For each training sample size N, Steps 1-3 were repeated 50 times to calculate the average out-of-sample accuracy (mean out-of-sample accuracy 1202 and standard deviation (Std) out-of-sample accuracy 1204). In FIG. 11a, the x-axis shows the number of samples included in the training sample I, representing 15 different sizes of training set; the y-axis is the average out-of-sample accuracy The rising trend of the mean of out-of-sample accuracies from 21% to 63% and the decreasing variance from 43% to 26% when increasing the training set size from 30 samples to 180 samples may suggest that that the model is projected to predict BNP level with higher accuracy and precision if more data are included in the model training.

32 patients in this study provided blood samples more than once in recurring visits, thus multiple IDs were associated with the same patient. These patients were separated into four categories based on the number of samples they provided ranging from 2 to 5 samples: there were 22, 6, 3, and 1 patient(s) who provided 2, 3, 4 and 5 samples, respectively.

To explore the trend of out-of-sample performance of the model when one patient gets tested repeatedly, the following experiment was performed:

(1) Divide the data into three parts: (a) for patients who have visited clinic K times such that K samples were provided, we randomly extracted K−1 samples; (b) rest of the samples from this patient; and (c) the rest of patient samples aside from the particular returning patient.

(2) Build a model with fixed CNN+TD structure considering (a)+(c) as the training samples.

(3) Calculate the out-of-sample accuracy considering (b) as the test sample.

(4) Repeat Steps 1-3 fifty times as simulation.

This experiment can demonstrate the simulated average BNP prediction accuracies with different number of returning visits. For each returning times K, Steps 1-3 were repeated 50 times to calculate the average out-of-sample accuracy (mean out-of-sample accuracy 1206 and standard deviation (Std) out-of-sample accuracy 1208). In FIG. 11b, the x-axis is the number of samples from the each returning patient included in the training; the y-axis represents the mean out-of-sample accuracies the test sample. Similarly, the mean of out-of-sample accuracies appeared to increase while the variance appeared to decrease as a patient returns for more test when previous test data were included in model training. Accordingly, the trained machine learning model may have the capacity to self-learn and may be used for periodic monitoring of BNP concentration for management of a health condition such as heart failure.

### Example 5—Two-Class Rule-Out & Rule-In Model Performance

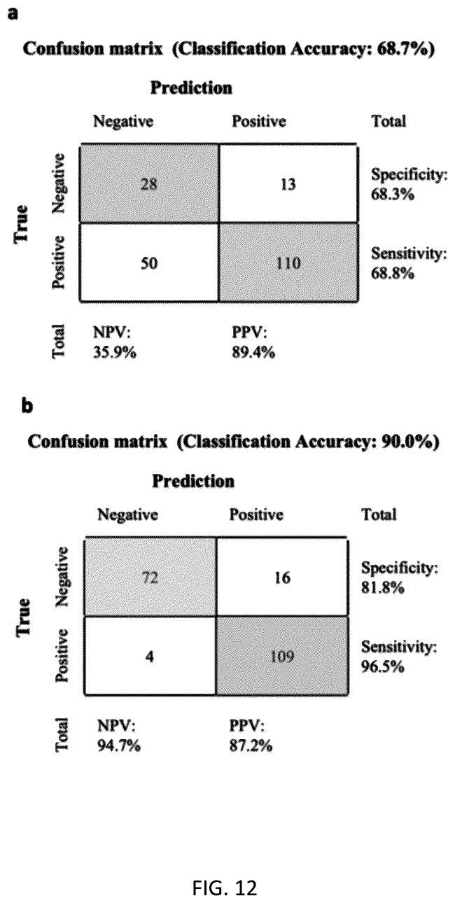

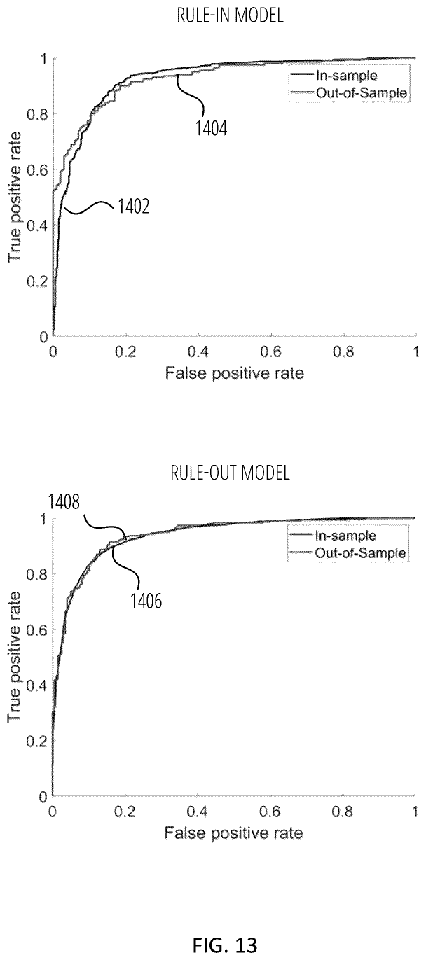

FIG. 12 and FIG. 13 show example data that may demonstrate the robustness of the trained machine learning model against overfitting problems.

FIG. 12 shows the out-of-sample prediction accuracies of sensor-level model for a two-class rule-out & rule-in model. In the rule-out model, only class 0 was considered as 0 (negative) and classes 1-3 were considered as 1 (positive). In the rule-in model, classes 0 and 1 were considered as 0 (negative) and classes 2 and 3 were considered as 1 (positive). The overall accuracies of out-of-sample rule-out and rule-in performance were 68.7% and 90.0%, respectively. The rule-in model may demonstrate improved performance in all aspects of test characteristics compared to the rule-out model, which may suggest that the two-class model may be better suited for rule-in application (i.e., classifying highly likely HF cases using 400 pg/mL as a cut-off). The number of data from class 0 was less than that of classes 1-3 in the rule-out model such that the unmatchable sample sizes may have reduced the rule-out model performance. The overall accuracies of in-sample performance for rule-out & rule-in model were 69.7% and 89.3%, respectively. As the overall accuracies of in-sample and out-of-sample performance may be considered close, the trained machine learning model may be considered robust against overfitting.

FIG. 13 shows the in-sample and out-of-sample ROC curves for two-class rule-out & rule-in model, where 1402 and 1404 are the in-sample and out-of-sample ROC curves for the rule-in model, respectively, and 1406 and 1408 are the in-sample and out-of-sample ROC curves for the rule-out model, respectively. Each pair of TPR and FPR on the ROC curve corresponds to one threshold value which may determine the point at which the classifier produces a positive or negative outcome. The AUC can provide an aggregate measure of performance across all possible classification thresholds. The rule-in model showed an AUC result around 0.9 which was considered excellent, whereas that of the rule-out model was rather fair, situating around 0.7. The trends of in-sample and out-of-sample results may suggest that the trained machine learning model may be considered robust against overfitting.

The following are non-limiting embodiments of the subject-matter disclosed herein.

Embodiment 1. A system for determining a concentration of an analyte in a fluid sample, the system comprising: at least one sensor operable to have a dynamic impedance spectrum dependent on the concentration of the analyte in the fluid sample when contacted by the fluid sample; a sensor driver operable to perform an impedance spectroscopy sweep on the at least one sensor at each of a plurality of time intervals to generate a set of temporal impedance spectra; at least one processor and at least one memory storing instructions operable by the at least one processor to process the set of temporal impedance spectra to generate the concentration of the analyte in the fluid sample.

Embodiment 2. The system of Embodiment 1, wherein: the plurality of time intervals comprises an initial time interval of 0 minutes after a contact time when the at least one sensor first contacts the fluid sample, and a final time interval of no more than 300 minutes after the contact time.

Embodiment 3. The system of Embodiments 1 or 2, wherein: the plurality of time intervals comprises at least 4 time intervals.

Embodiment 4. The system of any one of Embodiments 1 to 3, wherein: each impedance spectroscopy sweep is performed at at least 10 different frequencies between about 0.1 Hz to about 100 KHz.

Embodiment 5. The system of any one of Embodiments 1 to 4, wherein: the at least one sensor comprises a carbon nanotube thin film (CNT-TF) sensor.

Embodiment 6. The system of Embodiment 5, wherein: the CNT-TF is functionalizable to respond to the analyte.

Embodiment 7. The system of any one of Embodiments 1 to 6, wherein: the sensor driver comprises a potentiostat.

Embodiment 8. The system of any one of Embodiments 1 to 7, further comprising a sample cartridge enclosing the at least one sensor, the sample cartridge comprising: at least one sample chamber for containing the fluid sample, each sample chamber comprising a surface of at least one of the at least one sensor; a sample port; and microfluidic components fluidly coupling the sample port to the at least one sample chamber to communicate the fluid sample from the sample port to the at least one sample chamber.

Embodiment 9. The system of any one of Embodiments 1 to 8, further comprising: a sensor reader comprising: the sensor driver; a sample cartridge connector, wherein the sample cartridge further comprises a sensor reader connector electrically coupled with the at least one sensor, and the sample cartridge connector and the sensor reader connector are mutually configured for coupling the sample cartridge to the sensor reader to thereby electrically couple the sensor driver to the at least one sensor to perform the impedance spectroscopy sweep on the at least one sensor; a communication interface coupled to the memory to communicate the set of temporal impedance spectra for processing to generate the concentration of the analyte in the fluid sample; a power source to power at least the sensor driver and the memory; and a housing enclosing at least the sensor driver, the power source, and the memory.

Embodiment 10. The system of any one of Embodiments 1 to 9, wherein: processing the set of temporal impedance spectra comprises using at least one trained machine learning model on the set of temporal impedance spectra to generate the concentration of the analyte in the fluid sample.

Embodiment 11. The system of Embodiment 10, wherein: the at least one trained machine learning model comprises at least one convolutional neural network.

Embodiment 12. The system of Embodiment 11, wherein: the at least one convolutional neural network comprises at least one convolutional layer applying at least one kernel exhausting all linear combinations within each of the temporal impedance spectra.

Embodiment 13. The system of Embodiment 12, wherein: the at least one convolutional layer further applies at least one kernel exhausting all linear combinations of features across the set of temporal impedance spectra.

Embodiment 14. The system of Embodiment 13, wherein: the at least one convolutional neural network further comprises at least one down-sampling convolutional layer after the at least one convolutional layer.

Embodiment 15. The system of Embodiment 14, wherein: the at least one down-sampling convolutional layer comprises at least one max pooling layer.

Embodiment 16. The system of Embodiment 15, wherein: the at least one convolutional neural network further comprises at least one deep convolutional neural layer comprising a plurality of fully connected layers after the at least one down-sampling convolution layer.

Embodiment 17. The system of any one of Embodiments 10 to 16, wherein: the at least one trained machine learning model is trained by: obtaining at least one labelled set of temporal impedance spectra characteristic of the sensor exposed to the fluid sample; and
training the at least one machine learning model using the labeled set of temporal impedance spectra to generate the at least one trained machine learning model.

Embodiment 18. The system of any one of Embodiments 1 to 17, wherein: the analyte is B-type natriuretic peptide (BNP); and the fluid sample is a blood sample.

Embodiment 19. The system of Embodiment 18, wherein: the blood sample is a blood sample of a patient; and the instructions are further operable by the at least one processor to generate a likelihood category of heart failure based on the generated concentration of BNP in the blood sample.

Embodiment 20. A method for determining a concentration of an analyte in a fluid sample, the method comprising: receiving a set of temporal impedance spectra generated by performing an impedance spectroscopy sweep on at least one sensor at each of a plurality of time intervals, the at least one sensor contacting the fluid sample and having a dynamic impedance spectrum dependent on the concentration of the analyte in the fluid sample; and processing the set of temporal impedance spectra to generate the concentration of the analyte in the fluid sample.

Embodiment 21. The method of Embodiment 20, wherein: processing the set of temporal impedance spectra comprises using at least one trained machine learning model on the set of temporal impedance spectra to generate the concentration of the analyte in the fluid sample.

Embodiment 22. The method of Embodiment 21, wherein: the at least one trained machine learning model comprises at least one convolutional neural network.

Embodiment 23. The method of Embodiment 22, wherein: the at least one convolutional neural network comprises at least one convolutional layer applying at least one kernel exhausting all linear combinations within each of the temporal impedance spectra.

Embodiment 24. The method of Embodiment 23, wherein: the at least one convolutional layer further applies at least one kernel exhausting all linear combinations of features across the set of temporal impedance spectra.

Embodiment 25. The method of Embodiment 24, wherein: the at least one convolutional neural network further comprises at least one down-sampling convolutional layer after the at least one convolutional layer.

Embodiment 26. The method of Embodiment 25, wherein: the at least one down-sampling convolutional layer comprises at least one max pooling layer.

Embodiment 27. The method of Embodiment 26, wherein: the at least one convolutional neural network further comprises at least one deep convolutional neural layer comprising a plurality of fully connected layers after the at least one down-sampling convolution layer.

Embodiment 28. The method of any one of Embodiments 21 to 27, wherein: the at least one trained machine learning model is trained by: obtaining at least one labelled set of temporal impedance spectra characteristic of the sensor exposed to the fluid sample; and training the at least one machine learning model using the labeled set of temporal impedance spectra to generate the at least one trained machine learning model.

Embodiment 29. The method of any one of Embodiments 20 to 28, wherein: the plurality of time intervals comprises an initial time interval of 0 minutes after a contact time when the at least one sensor first contacts the fluid sample, and a final time interval of no more than 300 minutes after the contact time.

Embodiment 30. The method of any one of Embodiments 20 to 29, wherein: the plurality of time intervals comprises at least 4 time intervals.

Embodiment 31. The method of any one of Embodiments 20 to 30, wherein: each impedance spectroscopy sweep is performed at at least 10 different frequencies between about 0.1 Hz to about 100 KHz.

Embodiment 32. The method of any one of Embodiments 20 to 31, wherein: the sensor comprises a carbon nanotube thin film (CNT-TF) sensor functionalized to respond to the analyte.

Embodiment 33. The method of any one of Embodiments 20 to 32, further comprising: performing the impedance spectroscopy sweep.

Embodiment 34. The method any one of Embodiments 20 to 33, further comprising: displaying, transmitting, or storing the generated concentration of analyte in the fluid sample.

Embodiment 35. The method of any one of Embodiments 20 to 34, wherein: the analyte is B-type natriuretic peptide (BNP); and the fluid sample is a blood sample.

Embodiment 36. A method of diagnosing heart failure in a patient, the method comprising: performing the method of Embodiment 35, wherein the blood sample is a blood sample of the patient; and generating a likelihood category of heart failure based on the generated concentration of BNP in the blood sample.

Embodiment 37. The method of any one of Embodiments 20 to 36, further comprising: contacting the fluid sample with the sensor.

Embodiment 38. A non-transient computer-readable medium storing instructions operable to cause at least one processor to perform the method of any one of Embodiments 20 to 37.

Embodiment 39. A method of training a model to determine a concentration of an analyte in a fluid sample, the method comprising: obtaining a labelled set of temporal impedance spectra characteristic of a sensor exposed to the fluid sample, wherein a dynamic impedance spectrum of the sensor is dependent on the concentration of analyte in the fluid sample; and training at least one machine learning model using the labeled set of temporal impedance spectra to generate a trained machine learning model.

Embodiment 40. The method of Embodiment 3, wherein: the at least one trained machine learning model comprises at least one convolutional neural network.

Embodiment 41. The method of Embodiment 40, wherein: the at least one convolutional neural network comprises at least one convolutional layer applying at least one kernel exhausting all linear combinations within each of the temporal impedance spectra.

Embodiment 42. The method of Embodiment 41, wherein:

the at least one convolutional layer further applies at least one kernel exhaust-ing all linear combinations of features across the set of temporal impedance spectra.

Embodiment 43. The method of Embodiment 42, wherein:
the at least one convolutional neural network further comprises at least one down-sampling convolutional layer after the at least one convolutional layer.

Embodiment 44. The method of Embodiment 43, wherein:
the at least one down-sampling convolutional layer comprises at least one max pooling layer.

Embodiment 45. The method of Embodiment 44, wherein: the at least one convolutional neural network further comprises at least one deep convolutional neural layer comprising a plurality of fully connected layers after the at least one down-sampling convolution layer.

Embodiment 46. The method of any one of Embodiments 39 to 45, wherein: the analyte is B-type natriuretic peptide (BNP); and the fluid sample is a blood sample.

Embodiment 47. An apparatus for determining a concentration of an analyte in a fluid sample, the apparatus comprising: at least one sensor operable to have a dynamic impedance spectrum dependent on the concentration of the analyte in the fluid sample when contacted by the fluid sample; a sensor driver operable to perform an impedance spectroscopy sweep on the at least one sensor at each of a plurality of time intervals to generate a set of temporal impedance spectra; and a memory coupled to the sensor driver to store the set of temporal impedance spectra for processing to generate the concentration of the analyte in the fluid sample.

Embodiment 48. The apparatus of Embodiment 47, wherein: the plurality of time intervals comprises an initial time interval of 0 minutes af-ter a contact time when the at least one sensor first contacts the fluid sample, and a fi-nal time interval of no more than 300 minutes after the contact time.

Embodiment 49. The apparatus of Embodiment 47 or 48, wherein: the plurality of time intervals comprises at least 4 time intervals.

Embodiment 50. The apparatus of any one of claims 47 to 49, wherein: each impedance spectroscopy sweep is performed at at least 10 different fre-quencies between about 0.1 Hz to about 100 KHz.

Embodiment 51. The apparatus of any one of Embodiments 47 to 50, wherein: the at least one sensor comprises a carbon nanotube thin film (CNT-TF) sensor.

Embodiment 52. The apparatus of Embodiment 51, wherein: the CNT-TF is functionalizable to respond to the analyte.

Embodiment 53. The apparatus of any one of Embodiments 47 to 52, wherein: the sensor driver comprises a potentiostat.

Embodiment 54. The apparatus of any one of Embodiments 47 to 53, further comprising a sample cartridge enclosing the at least one sensor, the sample cartridge comprising: at least one sample chamber for containing the fluid sample, each sample chamber comprising a surface of at least one of the at least one sensor; a sample port; and microfluidic components fluidly coupling the sample port to the at least one sample chamber to communicate the fluid sample from the sample port to the at least one sample chamber.

Embodiment 55. The apparatus of any one of Embodiments 47 to 54, further comprising: a sensor reader comprising: the sensor driver; a sample cartridge connector, wherein the sample cartridge further comprises a sensor reader connector electrically coupled with the at least one sensor, and the sample cartridge connector and the sensor reader connector are mutually configured for coupling the sample cartridge to the sensor reader to thereby electrically couple the sensor driver to the at least one sensor to perform the impedance spectroscopy sweep on the at least one sensor; a communication interface coupled to the memory to communicate the set of temporal impedance spectra for processing to generate the concentration of the analyte in the fluid sample; a power source to power at least the sensor driver and the memory; and a housing enclosing at least the sensor driver, the power source, and the memory.

Embodiment 56. The apparatus of any one of Embodiments 47 to 55, wherein: the analyte is B-type natriuretic peptide (BNP); and the fluid sample is a blood sample.

Embodiment 57. An apparatus for determining a concentration of an analyte in a fluid sample, the device comprising: at least one processor and at least one memory storing instructions operable by the at least one processor: to process a set of temporal impedance spectra to generate the concentration of the analyte in the fluid sample, the set of temporal impedance spectra generated by performing an impedance spectroscopy sweep on at least one sensor at each of a plurality of time intervals, the at least one sensor contacting the fluid sample and having a dynamic impedance spectrum dependent on the concentration of the analyte in the fluid sample.

Embodiment 58. The apparatus of Embodiment 57, wherein: processing the set of temporal impedance spectra comprises using at least one trained machine learning model on the set of temporal impedance spectra to generate the concentration of the analyte in the fluid sample.

Embodiment 59. The apparatus of Embodiment 58, wherein: the at least one trained machine learning model comprises at least one convolutional neural network.

Embodiment 60. The apparatus of Embodiment 59, wherein: the at least one convolutional neural network comprises at least one convolutional layer applying at least one kernel exhausting all linear combinations within each of the temporal impedance spectra.

Embodiment 61. The apparatus of Embodiment 60, wherein: the at least one convolutional layer further applies at least one kernel exhausting all linear combinations of features across the set of temporal impedance spectra.

Embodiment 62. The apparatus of Embodiment 61, wherein: the at least one convolutional neural network further comprises at least one down-sampling convolutional layer after the at least one convolutional layer.

Embodiment 63. The apparatus of Embodiment 62, wherein: the at least one down-sampling convolutional layer comprises at least one max pooling layer.

Embodiment 64. The apparatus of Embodiment 63, wherein: the at least one convolutional neural network further comprises at least one deep convolutional neural layer comprising a plurality of fully connected layers after the at least one down-sampling convolution layer.

Embodiment 65. The apparatus of any one of Embodiments 58 to 64, wherein: the at least one trained machine learning model is trained by: obtaining at least one labelled set of temporal impedance spectra characteristic of the sensor exposed to the fluid sample; and training the at least one machine learning model using the labeled set of temporal impedance spectra to generate the at least one trained machine learning model.

Embodiment 66. The apparatus of any one of Embodiments 57 to 65, wherein: the plurality of time intervals comprises an initial time interval of 0 minutes after a contact time when the at least one sensor first contacts the fluid sample, and a final time interval of no more than 300 minutes after the contact time.

Embodiment 67. The apparatus of any one of Embodiments 57 to 66, wherein: the plurality of time intervals comprises at least 4 time intervals.

Embodiment 68. The apparatus of any one of Embodiments 57 to 67, wherein: each impedance spectroscopy sweep is performed at at least 10 different frequencies between about 0.1 Hz to about 100 KHz.

Embodiment 69. The apparatus of any one of Embodiments 57 to 68, wherein: the sensor comprises a carbon nanotube thin film (CNT-TF) sensor functionalized to respond to the analyte.

Embodiment 70. The apparatus of any one of Embodiments 57 to 69, further comprising a communication interface, wherein the instructions are further operable by the at least one processor to use the communication interface to receive the set of temporal impedance spectra.

Embodiment 71. The apparatus of any one of Embodiments 57 to 70, wherein: the analyte is B-type natriuretic peptide (BNP); and the fluid sample is a blood sample.

Embodiment 72. An apparatus for diagnosing heart failure in a patient, the apparatus comprising the apparatus of Embodiment 71, wherein: the blood sample is a blood sample of the patient; and the instructions are further operable by the at least one processor to generate a likelihood category of heart failure based on the generated concentration of BNP in the blood sample.

Embodiment 73. A system comprising the apparatus of any one of Embodiments 47 to 56 and the apparatus of any one of Embodiments 57 to 72.

So that the present disclosure may be more readily understood, certain terms are defined. Unless defined otherwise, all technical and scientific terms used herein have the same meaning as commonly understood by one of ordinary skill in the art to which embodiments of the invention pertain. While many methods and materials similar, modified, or equivalent to those described herein can be used in the practice of the embodiments of the present invention without undue experimentation, the preferred materials and methods are described herein.

All terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6, and decimals and fractions, for example, 1.2, 3.8, 1½, and 4¾. This applies regardless of the breadth of the range.

The terms “about” or “approximately” as used herein refer to variation in the numerical quantity that can occur, for example, through typical measuring techniques and equipment, with respect to any quantifiable variable, including, but not limited to, mass, volume, time, distance, voltage, and current. Further, given solid and liquid handling procedures used in the real world, there is certain inadvertent error and variation that is likely through differences in the manufacture, source, or purity of the ingredients used to make the compositions or carry out the methods and the like. The terms “about” and “approximately” also encompass these variations. Expressions which combine the terms “about” or “approximately” with one or more bounds of a range refer to a union of the bound modified by the term “about” or “approximately” as described above, and the range having the unmodified bound. Thus, for example, the expression “at least about X” means the union of “at least X” and “about X”. Similarly, “at most about Y” means the union of “at most Y” and “about Y”.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of”, or when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either”, “one of”, “only one of”, or “exactly one of”. “Consisting essentially of”, when used in the claims, shall have its ordinary meaning as used in the field of patent law.

Embodiments of the disclosed subject-matter are described herein using the auxiliary verb “may”. When used herein, unless required otherwise by the context of usage, the auxiliary verb “may” designates an embodiment of the disclosed subject-matter which possesses the addressed object without requiring necessarily that any other embodiment of the disclosed subject-matter possesses the addressed object. Thus, a statement such as “X may include Y” indicates that the disclosed subject-matter includes embodiments where X includes Y, without requiring that all disclosed embodiments include Y, and without excluding any other embodiments which do not include Y.

While the disclosed subject-matter may be embodied in many different forms, there are described in detail herein specific embodiments. The present disclosure is an exemplification of the principles of the disclosed subject-matter and is not intended to limit the disclosed subject-matter to the particular embodiments illustrated. Furthermore, the disclosed subject-matter encompasses any possible combination of some or all of the various embodiments mentioned herein. In addition the disclosed subject-matter encompasses any possible combination that also specifically excludes any one or some of the various embodiments mentioned herein.

In some instances, well-known hardware and software components, modules, and functions are shown in block diagram form in order not to obscure the invention. For example, specific details are not provided as to whether the embodiments described herein are implemented as a software routine, hardware circuit, firmware, or a combination thereof.

Some of the embodiments described herein include a processor and a memory storing computer-readable instructions executable by the processor. In some embodiments, the processor is a hardware processor configured to perform a predefined set of basic operations in response to receiving a corresponding basic instruction selected from a predefined native instruction set of codes. Each of the modules defined herein may include a corresponding set of machine codes selected from the native instruction set, and which may be stored in the memory.

Embodiments can be implemented as a software product stored in a machine-readable medium (also referred to as a computer-readable medium, a processor-readable medium, or a computer usable medium having a computer-readable program code embodied therein). The machine-readable medium can be any suitable tangible medium, including magnetic, optical, or electrical storage medium including a diskette, optical disc, memory device (volatile or non-volatile), or similar storage mechanism. The machine-readable medium can contain various sets of instructions, code sequences, configuration information, or other data, which, when executed, cause a processor to perform steps in a method according to an embodiment of the invention. Those of ordinary skill in the art will appreciate that other instructions and operations necessary to implement the described embodiments can also be stored on the machine-readable medium. Software running from the machine-readable medium can interface with circuitry to perform the described tasks.

In the preceding description, for purposes of explanation, numerous details are set forth in order to provide a thorough understanding of the embodiments. However, it will be apparent to one skilled in the art that these specific details are not required. In particular, it will be appreciated that the various additional features shown in the drawings are generally optional unless specifically identified herein as required. The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

What is claimed is:

1. A system for determining a concentration classification of an analyte in a fluid sample, the system comprising:
at least one sensor operable to have a dynamic impedance spectrum dependent on a concentration of the analyte in the fluid sample when contacted by the fluid sample;
a sensor driver operable to perform an impedance spectroscopy sweep on the at least one sensor at each of a plurality of time intervals to generate a set of temporal impedance spectra;
at least one processor and at least one memory storing instructions operable by the at least one processor to process the set of temporal impedance spectra to generate the concentration classification of the analyte in the fluid sample;
wherein the concentration classification of the analyte is the concentration or a concentration class;
wherein processing the set of temporal impedance spectra comprises using at least one trained machine learning model on the set of temporal impedance spectra to generate the concentration classification of the analyte in the fluid sample; and
wherein the at least one trained machine learning model is trained by:
obtaining at least one labelled set of temporal impedance spectra characteristic of the at least one sensor exposed to the fluid sample; and
training the at least one machine learning model using the labelled set of temporal impedance spectra to generate the at least one trained machine learning model.

2. The system of claim 1, wherein:
the plurality of time intervals comprises an initial time interval of 0 minutes after a contact time when the at least one sensor first contacts the fluid sample, and a final time interval of no more than 300 minutes after the contact time.

3. The system of claim 2, wherein:
the plurality of time intervals comprises at least 2 time intervals.

4. The system of claim 3, wherein:
each impedance spectroscopy sweep is performed at at least 2 different frequencies between about 0.1 Hz to about 100 KHz.

5. The system of claim 4, wherein:
the at least one sensor comprises a carbon nanotube thin film (CNT-TF) sensor.

6. The system of claim 5, wherein:
the CNT-TF is functionalizable to respond to the analyte.

7. The system of claim 6, wherein:
the sensor driver comprises a potentiostat.

8. The system of claim 7, further comprising a sample cartridge enclosing the at least one sensor, the sample cartridge comprising:
at least one sample chamber for containing the fluid sample, each sample chamber comprising a surface of at least one of the at least one sensor;
a sample port; and
microfluidic components fluidly coupling the sample port to the at least one sample chamber to communicate the fluid sample from the sample port to the at least one sample chamber.

9. The system of claim 8, further comprising:
a sensor reader comprising:
the sensor driver;
a sample cartridge connector, wherein the sample cartridge further comprises a sensor reader connector electrically coupled with the at least one sensor, and the sample cartridge connector and the sensor reader connector are mutually configured for coupling the sample cartridge to the sensor reader to thereby electrically couple the sensor driver to the at least one sensor to perform the impedance spectroscopy sweep on the at least one sensor;
a communication interface coupled to the memory to communicate the set of temporal impedance spectra for processing to generate the concentration classification of the analyte in the fluid sample;
a power source to power at least the sensor driver and the memory; and
a housing enclosing at least the sensor driver, the power source, and the memory.

10. The system of claim 9, wherein:
the at least one trained machine learning model comprises at least one convolutional neural network.

11. The system of claim 10, wherein:
the at least one convolutional neural network comprises at least one convolutional layer applying at least one kernel exhausting all linear combinations within each of the temporal impedance spectra.

12. The system of claim 11, wherein:
the at least one convolutional layer further applies at least one kernel exhausting all linear combinations of features across the set of temporal impedance spectra.

13. The system of claim 12, wherein:
the at least one convolutional neural network further comprises at least one down-sampling convolutional layer after the at least one convolutional layer.

14. The system of claim 13, wherein:
the at least one down-sampling convolutional layer comprises at least one max pooling layer.

15. The system of claim 14, wherein:
the at least one convolutional neural network further comprises at least one deep convolutional neural layer comprising a plurality of fully connected layers after the at least one down-sampling convolution layer.

16. The system of claim 15, wherein:
the analyte is B-type natriuretic peptide (BNP); and
the fluid sample is a blood sample.

17. The system of claim 16, wherein:
the blood sample is a blood sample of a patient; and
the instructions are further operable by the at least one processor to generate a likelihood category of heart failure based on the generated concentration classification of BNP in the blood sample.

18. A method for determining a concentration classification of an analyte in a fluid sample using the system of claim 1, the method comprising:
contacting the at least one sensor with the fluid sample;
operating the sensor driver to perform the impedance spectroscopy sweep on the least one sensor at each of the plurality of time intervals to generate the set of temporal impedance spectra; and
processing, by the at least one processor, the set of temporal impedance spectra to generate the concentration classification of the analyte in the fluid sample;
wherein processing the set of temporal impedance spectra comprises:
obtaining the at least one labelled set of temporal impedance spectra characteristic of the at least one sensor exposed to the fluid sample;
training the at least one machine learning model using the labelled set of temporal impedance spectra to generate the at least one trained machine learning model; and
generating the concentration classification of the analyte in the fluid sample using the at least one trained machine learning model.

19. The method of claim 18, wherein training the at least one machine learning model further comprises:
using a machine learning model comprising at least one hidden layer applying at least one kernel exhausting all linear combinations within each of the temporal impedance spectra and exhausting all linear combinations across the set of temporal impedance spectra.

20. The method of claim 18, further comprising:
generating a likelihood category of heart failure of a patient based on the generated concentration classification of the analyte in the fluid sample, wherein:
the analyte is B-type natriuretic peptide (BNP); and
the fluid sample is a blood sample of the patient.
